(19) United States
(12) Patent Application Publication
(10) Pub. No.: US 2013/0317364 A1
(43) Pub. Date: Nov. 28, 2013

(54) E-BRA AND METHODS FOR EARLY DETECTION OF BREAST TUMOR

(71) Applicants: Hansen Qi Yong Weng, Corona, NY (US); Shaam Periyapatna Sundhar, Princeton, NJ (US)

(72) Inventors: Hansen Qi Yong Weng, Corona, NY (US); Shaam Periyapatna Sundhar, Princeton, NJ (US)

(21) Appl. No.: 13/480,904

(22) Filed: May 25, 2012

(51) Int. Cl. A61B 8/08; A61B 6/00

(52) U.S. Cl. 600/442; 600/407

(57) ABSTRACT

An e-bra and a method of using the said e-bra for early detection of breast cancer are disclosed. The method requires women wear the e-bra once a week for ten minutes. The e-bra uses a high frequency electromagnetic wave or a low power laser to scan the breasts and the bounced echo strength is measured for identifying any size tumor in the breast tissue. When a tumor is detected, the e-bra sounds a warning signal to the woman and she needs to go to a doctor for further examination and evaluation.

### BACKGROUND OF THE INVENTION

1. Field of the Invention

The present invention relates to a medical device and methods of using the said medical device for early detection of breast cancer. More particularly, the present invention relates to an e-bra which can scan the breasts and identify any small size of tumor at a woman's home using a high frequency electromagnetic wave or a low power laser. The present invention also relates to methods using the said e-bra to scan the breast for the early detection of breast cancer.

According to “USA Today” and American Cancer Society (ACS), about 212,920 women were diagnosed with breast cancer from January 1, 200 to December 31 of the same year. Out of that about 40,600 died. The rest of women went through chemotherapy, costing hundreds of million dollars and untold human suffering. These numbers refer to the USA only, years after years these numbers are adding and cumulative total is becoming astronomical.

Breast cancer is the second leading cause of death of American women, and majority of death occur in the age group of 35 to 53. The early detection of breast cancer is almost a cure, because the localized small malign tumor may be removed easily before cancer spreads. When the tumor's diameter gets beyond 5 millimeter (mm) then the cancer cells migrate to different nodes and Organs. This makes cancer to spread to other parts of the body, then that may be too late.

The American Cancer Society recommends monthly self-examination after 20 years of age, physician examination once in three years up to 40 years of age, and once a year mammography after that age. The routine mammography is helpful in detecting breast cancer. However, a significant percentage of women do not go through the mammography test. For those who go, the insurance only pays once in a year. In the event if a doctor finds no breast tumor after the mammography and if the lady starts developing the cancer a week after the test then she may not find it until the following year. By that time it may be too late, the cancer may spread all over her body. Also, many women do not do their own self-examination, and it needs skills to find a small tumor in massive breast tissue.

Today in many devices a low power laser or a high frequency electromagnetic wave is used to bounce from an object. Then bounced echo strength is measured by an electronic circuitry. Examples of such devices includes “Aim & Shoot” distance measurement devices, Infra Red temperature guns, and routinely used Ultrasound machine in medical practice to exam different parts of the body. This type of technology has also been used for breast cancer detection, but it usually requires women to go to a medical clinic and the examination may also limited by the insurance company.

Therefore, what is desired is a simple and effective way to determine the breast cancer accurately at any given time in most convenient and economical way, at the privacy of women's home.

2. Description of Related Art

Some related prior inventions are disclosed as prior art herein. More specifically, by way of example:

The U.S. Pat. No. 4,509,368 discloses a method and apparatus for ultrasound tomography for use in clinical diagnosis, including breast cancer. The apparatus comprises of transmission transducers and reflection transducers to provide data capable of processing by a computational system to exam the internal structure of a body.

The U.S. Pub No. 2008/0249215 A1 disclosed a mammographic diagnostic apparatus using ultrasound probe.

### SUMMARY OF THE INVENTION

In an exemplary embodiment of the present invention, there is disclosed an e-bra and methods using the said e-bra for early detection of breast cancer. The two cups of the e-bra are formed by a fine wire mesh, and the two cups are sandwiched between two fine cloths to form a comfortable to wear bra. A high frequency electromagnetic wave or a low power laser directed towards the breast moves from one tiny square of the mesh to the next and the bounced echo strength is measured. When a tumor is detected, an electrical signal is sent to a tiny speaker and it beeps. The e-bra is powered with a small size battery and all the tiny speaker, battery, micro processor chips and circuits are incorporated in the strips of the e-bra. In an exemplary embodiment of the present invention, there is also disclosed a method using the said e-bra for early detection of breast cancer. The method comprises wearing the e-bra once a week for ten minutes to let the e-bra scan the breast and detect any size of tumors in the breast tissue and if the tiny speaker beeps the woman must go to doctors for further examination and evaluation.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a front view of the bra system.

FIG. 2 is a rear view of the bra system.

FIG. 3 depicts a feature on the front of the bra system.

FIG. 4 shows a side view of the bra system and shows key parts of the system itself.

### DESCRIPTION OF THE PREFERRED EMBODIMENT

In the present invention there are disclosed an e-bra and methods of using the said e-bra for early detection of the breast cancer. The two cups of the e-bra are formed by a fine wire mesh. A high frequency electromagnetic wave or a low power laser directed towards the breast moves from one tiny square of the mesh to the next and the bounced echo strength is measured. If, within a small variation, the echo strength is consistent, it indicates the healthy breast tissue. Whenever there is a pre-determined significant change in echo strength then an electronic signal is sent to a tiny speaker which makes it to sound a series of beeps. The e-bra is powered with a small size battery and all the tiny speaker, battery, micro processor chips and circuits are incorporated in the strips of the e-bra. In the present invention, there is also disclosed a method using the said e-bra for early detection of breast cancer. The method comprises wearing the e-bra once a week for ten minutes to let the e-bra scan the breast and detect any size of tumors in the breast tissue and if the tiny speaker beeps the woman must go to doctors for further examination and evaluation.

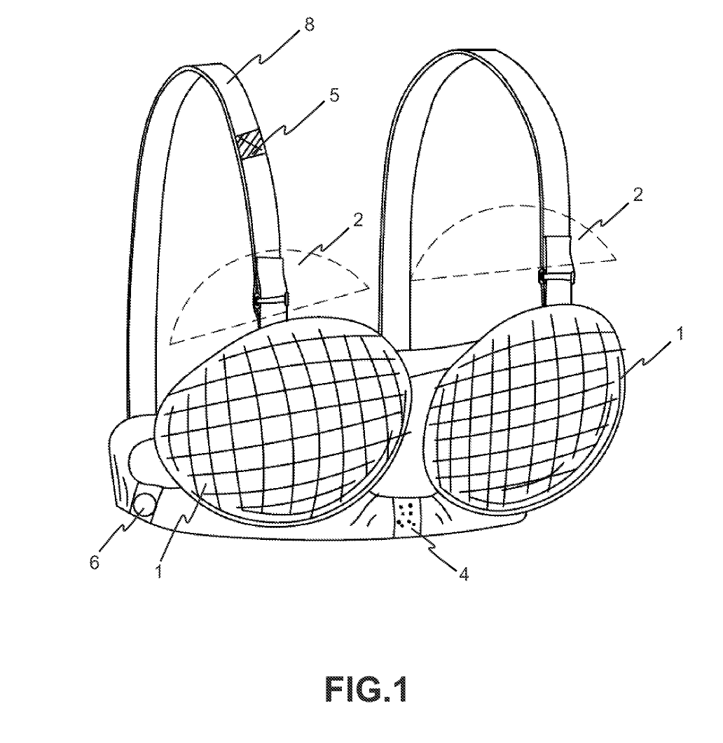

Referring to FIG. 1, the e-bra comprises two cups formed by a fine wire mesh 1 with tiny squares, an emitting module 3 to emitting a high frequency electromagnetic wave or a low power laser towards to the breasts moving from one tiny square of the mash to the next, a measure module 4 to measure the bounced echo strength, a tiny speaker 5 connected to the measure module, a battery 6 powering the e-bra.

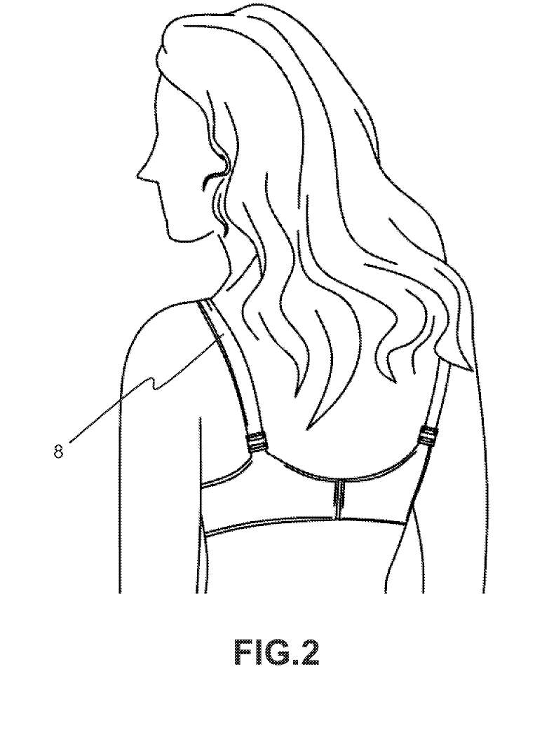

Referring to FIG. 2, the e-bra attaches in the rear with two straps 8 and center clasp.

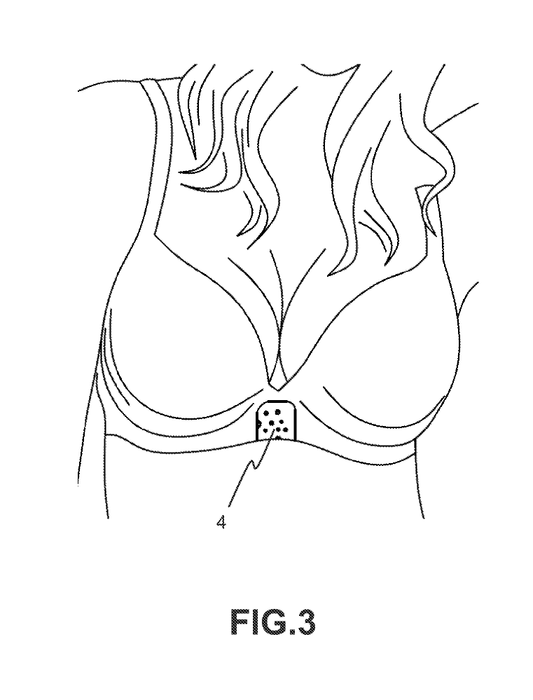

Referring to FIG. 3, the e-bra is shown from a front view where the measure module 4 to measure the bounced echo strength is clearly depicted.

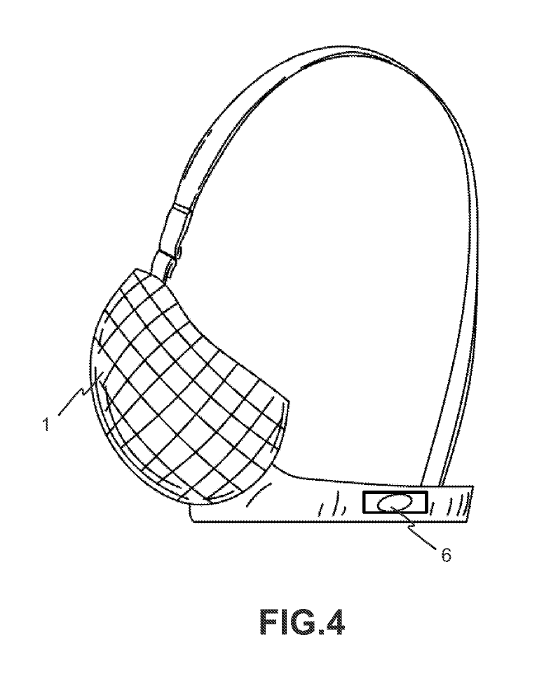

Referring to FIG. 4, the e-bra has a battery 6 which is worn on the e-bra itself on the side of the e-bra directly behind the wire mesh along the side strap of the e-bra.

In another embodiment of the present invention, the two cups of the e-bra are formed by a fine wire mesh and the said two cups are sandwiched between two cloths 2 to form a comfortable to ware bra. Yet in another embodiment of the present invention, the size of the tiny square of the mesh is 1 mm².

In another embodiment of present invention, the battery 6 is a 1.5 V triple A size Lithium battery and in another embodiment the said battery is placed in the bottom strip. Yet in another embodiment of the present invention, the battery is replaceable.

In another embodiment of the present invention, the emitting module 4 and measuring module 5 are placed in the bottom strip. In another embodiment of the present invention, the emitting module emits a low power laser. In another embodiment of the present invention, the emitting module emits an ultra sound wave. In another embodiment of the present invention, the said laser or ultra sound wave moves from one tiny square of the mesh to the next in each row or in each column.

In another embodiment of the present invention, the tiny speaker is embedded on to one of the shoulder strips.

Still in another embodiment of the present invention, there is disclosed a method of early detection of breast cancer by using the said e-bra. The method comprises wearing the e-bra once a week for ten minutes to let the e-bra scan the breast tissue. If there are no beeps then the woman just needs to repeat the test in the following week. If the e-bra beeps, the woman must go to a doctor for further examination and evaluation.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. An e-bra comprising:
a) two cups formed by a fine wire mesh;
b) an emitting module emitting a high frequency electromagnetic wave or a low power laser towards the breasts moving from one tiny square of the said mesh to the next;
c) an measuring module measuring the bounced echo strength of the said electromagnetic wave or laser bounced from the breasts;
d) a tiny speaker connected to the said measuring module; and
e) a battery powering the e-bra.

2. An e-bra of claim 1, wherein the two cups are sandwiched between two cloths to form a comfortable wear bra.

3. An e-bra of claim 1, wherein the emitting module emits a low power laser.

4. An e-bra of claim 1, wherein the emitting module emits an ultra sound wave.

5. An e-bra of claim 1, wherein the said emitting module and measuring module are placed in the bottom strip of the bra.

6. An e-bra of claim 1, the said high frequency electromagnetic wave or the said low power laser moving from one tiny square of the mesh to the next in a row or in a column.

7. An e-bra of claim 1, wherein the battery is a 1.5 V triple A size Lithium battery.

8. An e-bra of claim 1, wherein the battery is replaceable.

9. An e-bra of claim 1, wherein the tiny speaker is embedded in one of the shoulder strips of the bra.

10. An e-bra of claim 1, comprising:
a) two cups formed by a fine wire mesh of which the size of the tiny square is about 1 mm², and the two cups are sandwiched between two clothes to form a comfortable wear bra;
b) an emitting module emitting a lower power laser or an ultra sound wave towards to the breasts of the wearer from one square to the next in a row or in a column, the said emitting module is placed in the bottom strip;
c) an measuring module measuring the bounced echo strength of the laser or ultra sound wave and the said measuring module is placed in the bottom strip;
d) a tiny speaker connected to the said measuring module and the said speaker is embedded on to one of the shoulder strips;
e) a 1.5 V triple A size Lithium battery placed in the bottom strip and the said battery is replaceable; and
f) an on/off switch or button to turn on/off the said e-bra.

11. A method for early detection of breast cancer using presently disclosed e-bra, comprising of:
a) wearing the presently disclosed e-bra once a week;
b) let the e-bra scan the breasts of the wearer for 10 minutes;
c) if the tiny speaker in the e-bra sounds no warning signals, the wearer repeats the test in the following week;
d) if the tiny speaker in the e-bra sounds warning signals, the wearer needs to go to a doctor for further examination and evaluation.

12. A method for early detection of breast cancer of claim 11, wherein the e-bra comprising of:
a) two cups formed by a fine wire mesh;
b) an emitting module emitting a high frequency electromagnetic wave or a low power laser towards the breasts moving from one tiny square of the said mesh to the next;
c) an measuring module measuring the bounced echo strength of the said electromagnetic wave or laser bounced from the breasts;
d) a tiny speaker connected to the said measuring module; and
e) a battery powering the e-bra.

13. A method for early detection of breast cancer of claim 11, wherein the e-bra comprising of:
a) two cups formed by a fine wire mesh of which the size of the tiny square is about 1 mm², and the two cups are sandwiched between two clothes to form a comfortable wear bra;
b) an emitting module emitting a lower power laser or an ultra sound wave towards to the breasts of the wearer from one square to the next in a row or in a column, the said emitting module is placed in the bottom strip;
c) an measuring module measuring the bounced echo strength of the laser or ultra sound wave and the said measuring module is placed in the bottom strip;
d) a tiny speaker connected to the said measuring module and the said speaker is embedded on to one of the shoulder strips;
e) a 1.5 V triple A size Lithium battery placed in the bottom strip and the said battery is replaceable; and
f) an on/off switch or button to turn on/off the said e-bra.
